(19) United States
(12) Patent Application Publication
(10) Pub. No.: US 2012/0078089 A1
(43) Pub. Date: Mar. 29, 2012

(54) METHOD AND APPARATUS FOR GENERATING MEDICAL IMAGES

(75) Inventors: SCOTT DAVID WOLLENWEBER, WAUKESHA, WI (US); ALEXANDER GANIN, MILWAUKEE, WI (US)

(73) Assignee: GENERAL ELECTRIC COMPANY, SCHENECTADY, NY (US)

(21) Appl. No.: 12/888,960

(22) Filed: Sep. 23, 2010

(51) Int. Cl. A61B 5/05; G01T 1/164

(52) U.S. Cl. 600/427; 250/363.03

(57) ABSTRACT

A method for generating a hybrid imaging volume includes acquiring a Positron Emission Tomography (PET) imaging dataset of an object using a PET imaging system, the PET imaging dataset including at least one motion affected portion and at least one non-motion affected portion, identifying a motion affected portion of the PET imaging dataset, motion correcting the identified portion of the PET imaging dataset to generate a hybrid portion, and constructing a hybrid PET image volume using the hybrid portion and the at least one non-motion affected portion. A system for implementing the method is also described herein.

### BACKGROUND OF THE INVENTION

The subject matter disclosed herein relates generally to imaging systems, and more particularly, to an apparatus and method for generating medical images.

Multi-modality imaging systems exist that scan using different modalities, for example, Computed Tomography (CT), Magnetic Resonance Imaging (MRI), Positron Emission Tomography (PET), and Single Photon Emission Computed Tomography (SPECT). During operation, the image quality of conventional imaging systems may be affected by the motion of the object being imaged. In particular, motion of the imaged object can degrade the image quality. More specifically, image artifacts are produced by movement of the object during image acquisition. Respiratory motion is a common source of involuntary motion in mammals (e.g., people and animals) encountered in medical imaging systems. The respiratory motion may lead to errors during image review, such as when a physician is determining the size of a lesion, determining the location of the lesion, or quantifying the lesion.

To correct for motion related imaging artifacts, at least one conventional imaging system utilizes various techniques to correct for motion related imaging artifacts. However, the quantity of data produced by utilizing the various motion correction techniques is typically relatively large. Specifically, the various known techniques generate more data than is typically required by a physician to assess the medical condition from the imaged object. Accordingly, the physician is required to view all the data, including the motion-corrected data, to determine which portions of the data best represent the medical condition being diagnosed.

### BRIEF DESCRIPTION OF THE INVENTION

In one embodiment, a method for generating a hybrid imaging volume is provided. The method includes acquiring a Positron Emission Tomography (PET) imaging dataset of an object using a PET imaging system, the PET imaging dataset including at least one motion affected portion and at least one non-motion affected portion, identifying a motion affected portion of the PET imaging dataset, motion correcting the identified portion of the PET imaging dataset to generate a hybrid portion, and constructing a hybrid PET image volume using the hybrid portion and the at least one non-motion affected portion.

In another embodiment, a method of improving the quality of a medical image is provided. The method includes generating a plurality of gated Positron Emission Tomography (PET) images, motion correcting the gated PET images using a PET reference gate to generate a hybrid PET series of images, selecting at least one Computed Tomography (CT) image having the same respiratory phase as the gated PET images stored in the PET reference bin, and constructing at least one PET image volume using the hybrid PET series of images.

In a further embodiment, a multi-modality imaging system is provided. The imaging system includes a first modality unit, a second modality unit, and a computer operationally coupled to the first and second modality units. The is programmed to acquire a Positron Emission Tomography (PET) imaging dataset of an object using a PET imaging system, the PET imaging dataset including at least one motion affected portion and at least one non-motion affected portion, identify the motion affected portion of the PET imaging dataset, motion correct the identified portion of the PET imaging dataset to generate a hybrid portion, and construct a hybrid PET image using the hybrid portion and the at least one non-motion affected portion.

### BRIEF DESCRIPTION OF THE DRAWINGS

FIG. 1 is a pictorial view of an exemplary multi-modality imaging system formed in accordance with various embodiments.

FIG. 2 is a flowchart illustrating an exemplary method for generating at least one of a hybrid PET image or a hybrid CT image in accordance with various embodiments.

FIG. 3 is an exemplary CT scout scan image that may be generated using various scanning protocols in accordance with various embodiments.

FIG. 4 is an exemplary image generated in accordance with various embodiments.

FIG. 5 is an exemplary PET image generated in accordance with various embodiments.

FIG. 6 is another exemplary image generated in accordance with various embodiments.

FIG. 7 is a block diagram illustrating a plurality of exemplary bins that may be formed in accordance with various embodiments.

### DETAILED DESCRIPTION OF THE INVENTION

The foregoing summary, as well as the following detailed description of various embodiments, will be better understood when read in conjunction with the appended drawings. To the extent that the figures illustrate diagrams of the functional blocks of the various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Also as used herein, the phrase “reconstructing an image” is not intended to exclude embodiments of the present invention in which data representing an image is generated, but a viewable image is not. Therefore, as used herein the term “image” broadly refers to both viewable images and data representing a viewable image. However, many embodiments generate, or are configured to generate, at least one viewable image.

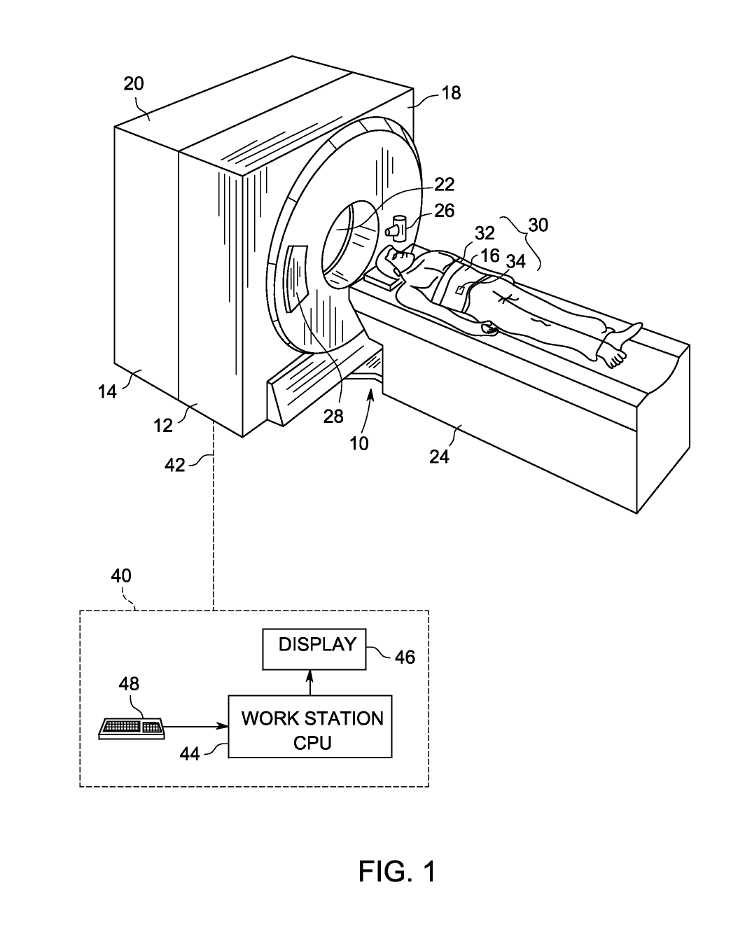

Various embodiments described herein provide a multi-modality imaging system 10 as shown in FIG. 1. The multi-modality imaging system 10 may be any type imaging system, for example, different types of medical imaging systems, such as a Positron Emission Tomography (PET), a Single Photon Emission Computed Tomography (SPECT), a Computed Tomography (CT), an ultrasound system, Magnetic Resonance Imaging (MRI) or any other system capable of generating diagnostic images. The various embodiments are not limited to multi-modality medical imaging systems, but may be used on a single modality medical imaging system such as a stand-alone PET imaging system or a stand-alone CT imaging system, for example. Moreover, the various embodiments are not limited to medical imaging systems for imaging human subjects, but may include veterinary or non-medical systems for imaging non-human objects, etc.

Referring to FIG. 1, the multi-modality imaging system 10 includes a first modality unit 12 and a second modality unit 14. The two modality units enable the multi-modality imaging system 10 to scan an object or patient 16 in a first modality using the first modality unit 12 and to scan the patient 16 in a second modality using the second modality unit 14. The multi-modality imaging system 10 allows for multiple scans in different modalities to facilitate an increased diagnostic capability over single modality systems. In one embodiment, multi-modality imaging system 10 is a PET/CT imaging system 10, e.g. the first modality 12 is a CT imaging system and the second modality 14 is a PET imaging system. The imaging system 10 is shown as including a gantry 18 that is associated with the CT imaging system 12 and a gantry 20 that is associated with the PET imaging system 14. During operation, the patient 16 is positioned within a central, opening 22, defined through the imaging system 10, using, for example, a motorized table 24.

The gantry 18 includes an x-ray source, 26 that projects a beam of x-rays toward a detector array 28 on the opposite side of the gantry 18. The detector array 28 is formed by a plurality of detector rows (not shown) including a plurality of detector elements which together sense the projected x-rays that pass through the patient 16. Each detector element produces an electrical signal that represents the intensity of an impinging x-ray beam and hence allows estimation of the attenuation of the beam as the beam passes through the patient 16. During a scan to acquire x-ray attenuation data, the gantry 18 and the components mounted thereon rotate about a center of rotation. Additionally, the PET imaging system includes a detector (not shown) that is configured to acquire emission data.

The imaging system 10 also includes at least one motion sensor 30 that is adapted to detect and transmit information that is indicative of the motion of the patient 16. In one embodiment, the motion sensor 30 may be embodied as a belt-type motion sensor 32 that is adapted to extend at least partially around the patient 16. Optionally, the motion sensor 30 may be embodied as a motion sensor 34 that is adapted to be secured to a predetermined position on the patient 16. It should be realized that although two different motion sensors are described, that the imaging system 10 may include other types of motions sensors to generate motion related information of the patient 16.

The imaging system 10 also includes an operator workstation 40. During operation, the motorized table 24 moves the patient 16 into the central opening 22 of the gantry 18 and/or 20 in response to one or more commands received from the operator workstation 40. The workstation 40 then operates the first and second modalities 12 and 14 to both scan the patient 16 and acquire attenuation and/or emission data of the patient 16. The workstation 40 may be embodied as a personal computer (PC) that is positioned near the imaging system 10 and hard-wired to the imaging system 10 via a communication link 42. The workstation 40 may also be embodied as a portable computer such as a laptop computer or a hand-held computer that transmits information to, and receives information, including motion information, from the imaging system 10. Optionally, the communication link 42 may be a wireless communication link that enables information to be transmitted to or from the workstation 40 to the imaging system 10 wirelessly. In operation, the workstation 40 is configured to control the operation of the imaging system 10 in real-time. The workstation 40 is also programmed to perform medical image diagnostic acquisition and reconstruction processes described herein.

The operator workstation 40 includes a central processing unit (CPU) or computer 44, a display 46, and an input device 48. As used herein, the term “computer” may include any processor-based or microprocessor-based system including systems using microcontrollers, reduced instruction set computers (RISC), application specific integrated circuits (ASICs), field programmable gate array (FPGAs), logic circuits, and any other circuit dr processor capable of executing the functions described herein. The above examples are exemplary only, and are thus not intended to limit in any way the definition and/or meaning of the term “computer”. In the exemplary embodiment, the computer 44 executes a set of instructions that are stored in one or more storage elements or memories, in order to process information received from the first and second modalities 12 and 14. The storage elements may also store data or other information as desired or needed. The storage element may be in the form of an information source or a physical memory element located within the computer 44.

The set of instructions may include various commands that instruct the computer 44 as a processing machine to perform specific operations such as the methods and processes of the various embodiments described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

The computer 44 connects to the communication link 42 and receives inputs, e.g., user commands, from the input device 48. The input device 48 may be, for example, a keyboard, mouse, a touch-screen panel, and/or a voice recognition system, etc. Through the input device 48 and associated control panel switches, the operator can control the operation of the CT imaging system 12 and the PET imaging system 14 and the positioning of the patient 16 for a scan. Similarly, the operator can control the display of the resulting image on the display 46 and can perform image-enhancement functions using programs executed by the computer 44.

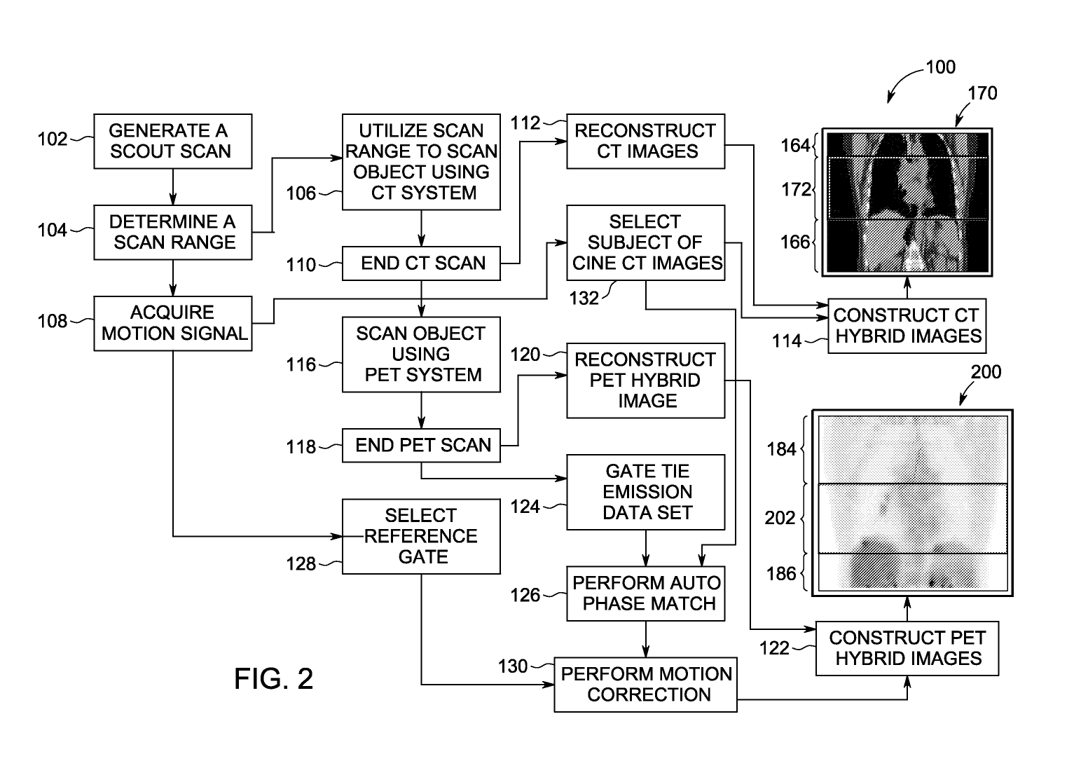

FIG. 2 is a simplified block diagram of an exemplary method 100 for generating a hybrid PET image and/or a hybrid CT image. Hybrid as used herein, means an image that includes a mixed set of image data, such as gated data and ungated or static data. Specifically, the ungated data is not motion corrected. Whereas, the gated data may be motion corrected. It should be noted that gated data adds a fourth dimension to the image data, such as time or gate/bin number. In the exemplary embodiment, the method 100 may be performed by the imaging system 10 shown in FIG. 1 and may be implemented by the computer 44 in accordance with various embodiments. In some embodiments, the method 100 is utilized to generate both a three-dimensional (3D) CT image and a 3D PET image, wherein each of the 3D CT image and the 3D PET image includes motion corrected information and non-motion corrected information. Thus, the method 100 reduces the quantity of information that a physician is required to review for a medical diagnosis. The method 100 also provides a hybrid CT image and a hybrid PET image that may be utilized by the physician to identify both structural and physiological conditions.

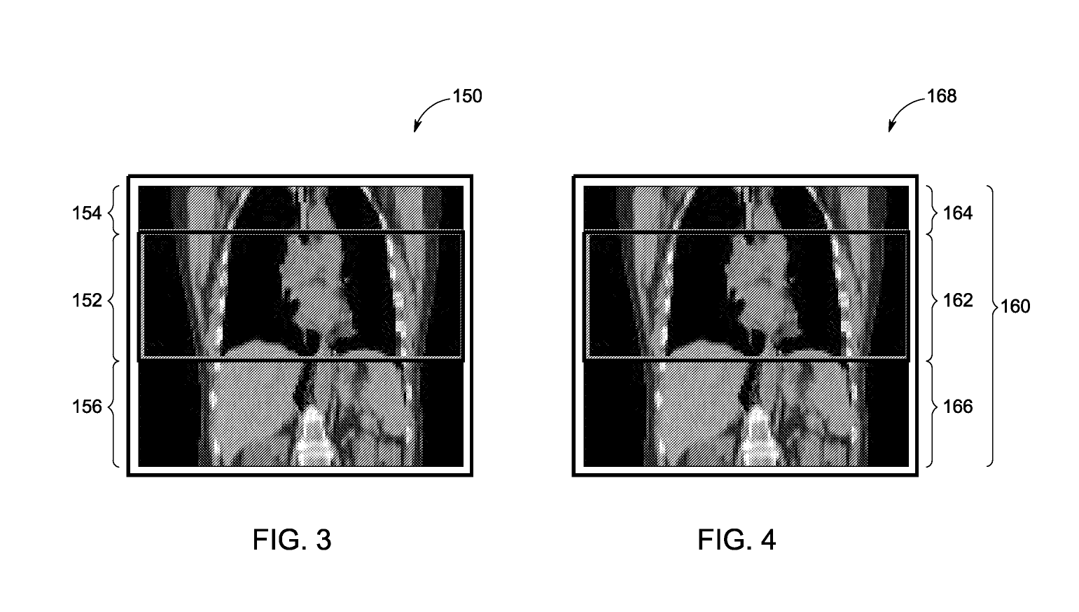

At 102 at least one scout scan of the patient 16 is performed to generate a scout scan image 150 shown in FIG. 3. In the exemplary embodiment, the scout scan is performed by the CT imaging system 12 over a relatively short duration to produce a single 2D image such as, for example, the image 150, that is similar to an x-ray of the patient 16. The range over which the internal motion information of the patient 16 is to be measured may be determined by viewing the image 150 generated by the scout scan.

At 104, a scan range that includes a volume of interest to be motion corrected is selected. In one embodiment, an exemplary volume of interest 152 (shown in FIG. 3) is selected manually by the operator atter reviewing the scout scan image 150. Optionally, the volume of interest 152 may be selected automatically by the imaging system 10 by comparing the scan data utilized to generate the scout image 150 to historical scan data. In another embodiment, the volume of interest 152 may be manually selected by the operator based on a priori operator information. For example, the operator may have knowledge where motion typically occurs during the imaging procedure or is more likely to occur. Based on this information, the operator may then manually select, by highlighting the selected volume for example, the volume of interest 152 to be motion corrected.

Referring again to FIG. 2, at 106 the scan range determined at 104 is utilized to scan the patient 16 using the CT system 12 to acquire transmission data that represents the patient 16. For example, as discussed above, typically only a portion of the information acquired at 106 is typically affected by patient motion. Thus, in the exemplary embodiment, scanning at 106 includes scanning the patient 16 using more than one scanning protocol. For example, referring again to FIG. 3, based on the scout scan, or other information, it may be determined that a second volume of interest 154 and a third volume of interest 156 have little or no motion that may affect image quality. Thus, the second and third volumes of interest 154 and 156 may be scanned using a first scanning protocol that generates less information and/or extends over a shorter time duration.

However, as discussed above, the selected volume of interest 152 has been predetermined to be more likely affected by motion, thus the selected volume of interest 152 may be scanned using a second different scanning protocol. For example, the selected volume of interest 152 may be scanned at a higher resolution and/or over a longer time duration and/or with respiratory gating to generate additional more information than is acquired using the first scanning protocol. It should be realized that although the selected volume of interest 152 is shown as being axially located between the second and third volumes of interest 154 and 156, the selected volume of interest 152 may be located anywhere within the overall volume of interest produced by the scanning procedure. Moreover, although only one volume of interest 152 is shown, it should be realized that multiple of volumes of interest, each affected by motion, may be selected.

At 108, a signal indicative of motion of the selected volume of interest 152 of the patient 16 is obtained. The motion signal may be obtained during the CT imaging scan at 106, during a related PET imaging scan, or during any other medical imaging system scanning procedure. Optionally, the motion signal may be obtained from a database of previous medical examination procedures. In the exemplary embodiment, the motion signal is obtained using the motion sensor 30 shown in FIG. 1. Optionally, the motion signal may be obtained from information saved in a memory device located in the computer 44. In the exemplary embodiment, the motion signal is representative of the motion of the patient 16 within the selected volume of interest 152.

At 110, the CT scanning of patient 16 is completed. As a result of the scanning procedure described at 106, an initial CT imaging dataset 160 (shown in FIG. 4) of the patient 16 is generated. For example, FIG. 4 is an exemplary CT image 168 generated from different types of data within the initial CT imaging dataset 160. As shown in FIG. 4, the initial CT imaging dataset 160 includes at least one portion 162 of information that reflects information acquired during scanning of at least one region that may be affected by motion, e.g. the selected volume of interest 152. Moreover, the initial CT imaging dataset 160 may include portions of information 164 and 166 that are not affected by motion, e.g. volumes of interest 154 and 156.

At 112, in the exemplary embodiment, the CT imaging dataset 160 acquired at 106 is reconstructed. For example, the helical cine CT images for portions 164 and 166, that are not affected by motion, e.g. acquired from volumes of interest 154 and 156, may be reconstructed to form a portion of the hybrid image 170. Thereafter, at 114, the information acquired at 106 may be utilized directly to construct the hybrid CT image 170 that includes the 3D information for portions 164 and 166 and a hybrid portion 172 that represents the portion 162 after motion correction is performed on the portion 162. The method of performing the motion correction on the portion 162 to generate the hybrid portion 172 is discussed in more detail below.

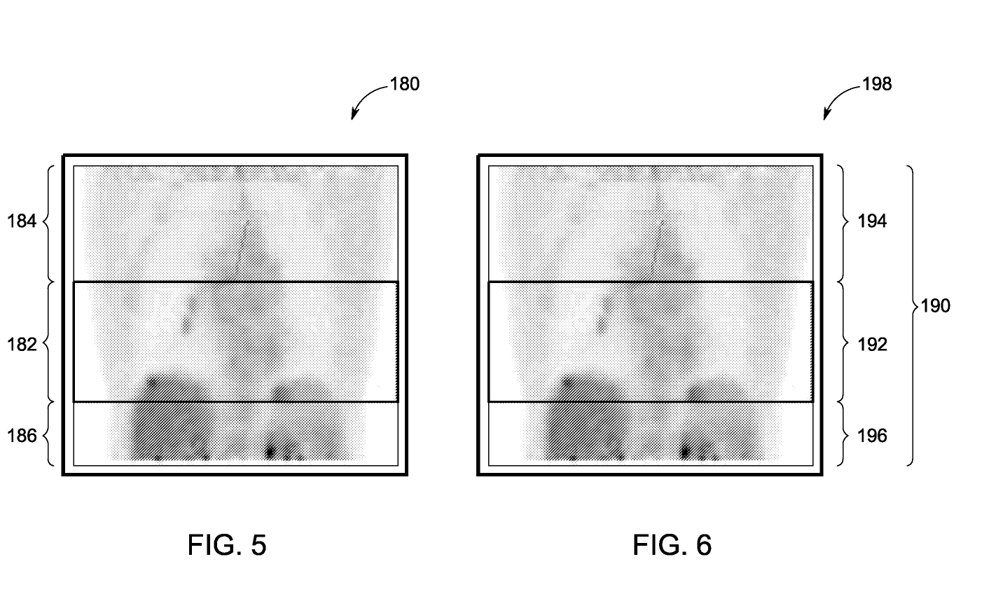

At 116, the patient 16 is scanned using the PET imaging system 14 to acquire emission data of the patient 16. In the exemplary embodiment, the selected volume of interest 152 is again utilized to perform the scanning procedure at 116. For example, as discussed above, only a portion of the information acquired at 106 is typically affected by motion. Thus, in the exemplary embodiment, scanning at 116 includes scanning the patient 16 using more than one scanning protocol. For example, FIG. 5 is an exemplary PET image 180 generated using the various scanning protocols that may be used by the PET imaging system 14. As discussed above, based on the CT scout scan image 150, or other information, it may be determined that a second volume interest 184 and a third volume of interest 186 have little or no motion that may affect image quality of the PET image 180. Thus, the second and third volumes of interest 184 and 186 may be scanned using a first imaging protocol that generates less information and/or extends over a shorter time duration.

However, as discussed above, a selected volume of interest 182, which typically is at the same axial location as the selected volume of interest 152 in the CT imaging dataset, has been predetermined to be more likely affected by motion, thus the selected volume of interest 182 may be scanned using a second different imaging protocol. For example, the selected volume of interest 182 may be scanned at a higher resolution and/or over a longer time period and/or with respiratory gating to generate more information than the first imaging protocol. It should be realized that although the selected volume of interest 182 is shown as being axially located between the second and third volumes of interest 184 and 186, the selected volume of interest 182 may be located anywhere within the overall volume of interest produced by the scanning procedure. Moreover, although only one volume of interest 182 is shown, it should be realized that multiple of volumes of interest, each affected by motion, may be selected.

In various embodiments, a portion of the emission data acquired during the scanning at 116 may be acquired in a list of events, a mode commonly referred to as list mode. Further, another portion of, the emission data may be acquired in a sinogram mode. The list mode generally refers to an acquisition mode in which each annihilation event is stored sequentially in a list mode file. The sinogram mode generally refers to an acquisition mode in which annihilation events, optionally having an identical Time-of-Flight (TOF), are stored in sinograms in an (radius from axis, angle) format. In one embodiment, a portion of the emission data may be acquired in the list mode for regions outside the selected volume of interest 182 and a portion of emission data is acquired in the sinogram mode for other portions, such as for example, the second and third regions of interest 184 and 186. In another embodiment, a portion of the emission data may be acquired in the list mode for regions outside the volume of interest. Further, a portion of the emission data may be acquired simultaneously, or concurrently, both in list mode, and sinogram mode for the volume of interest. In yet another embodiment of the invention, a portion of the emission data may be acquired in the list mode for every x annihilation event, where x is a positive number greater than one.

Referring again to FIG. 2, at 118, the scanning of the patient 16 is completed. As a result of the scanning procedure described at 116, an initial PET imaging dataset 190 of the patient 16 is generated.

For example, FIG. 6 is an exemplary PET image 198 generated using different types of data within the initial PET imaging dataset 190. As shown in FIG. 6, the initial PET imaging dataset 190 includes at least one portion 192 of information that reflects information acquired during the scanning for the region that may be affected by motion, e.g. the selected volume of interest 182. Moreover, the imaging dataset 190 may include portions of information 194 and 196 that are not affected by motion, e.g. volumes of interest 184 and 186.

At 120, in the exemplary embodiment, a portion of the initial PET imaging dataset 190 acquired at 116 is utilized to reconstruct un-gated PET images 190 of the patient 16. For example, the emission information for portions 194 and 196, that are not affected by motion, e.g. volumes of interest 184 and 186, may be reconstructed to form a portion of the hybrid image 200. Thereafter, at 122, the information acquired at 116 may be utilized directly to construct the hybrid PET image 200 (shown in FIG. 6). The hybrid PET image 200 includes the 3D information for portions 194 and 196 acquired from the initial PET imaging dataset 190 and also includes a portion 202 that represents the portion 192 after motion correction 130 is performed on the portion 192.

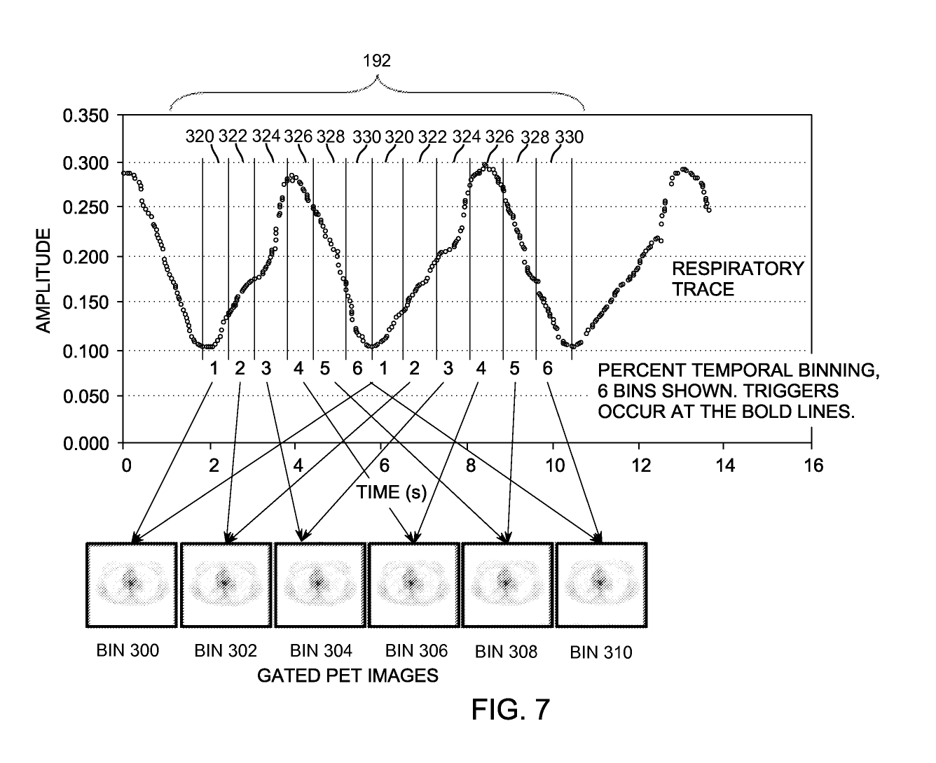

Referring again to FIG. 2, the method of performing the motion correction on the portion 192 to generate the hybrid PET portion 202 is now discussed. At 124, information that represents emission data within the PET volume 192 is binned or gated. As discussed above, the portion 192 represents emission information acquired during the scanning for the region that was pre-selected as potentially affected by motion, e.g. the selected volume of interest 182. In the exemplary embodiment, the emission data representing the portion 192 is binned into n bins. For example, FIG. 7 illustrates a plurality of bins numbered 300 . . . 310, i.e. n=6. In the exemplary embodiment, the emission data forming the portion 192 is then gated into the six bins numbered 300, 302, 304, 306, 308, and 310. However, it should be realized that the quantity of bins illustrated, in FIG. 7 is exemplary, and that during operation, fewer than six bins or more than six bins may be utilized. As such, each bin 300, 302, 304, 306, 308, and 310 includes approximately ⅙ of the total emission data within the portion 192.

For example, assuming that the total length of the PET scan to acquire emission data for the region 182 is three minutes, then the resulting portion of emission data 192 representing the region of interest 182 covers three minutes. Moreover, assuming that the emission data portion 192 is gated into six bins, then each respective bin includes approximately 30 seconds of emission data from the region of interest 182. Thus a first portion 320 of the emission data portion 192 is gated into the gate 300, a second portion 322 of the emission data portion 192 is gated into the gate 302, a third portion 324 of the emission data portion 192 is gated into the gate 304, a fourth portion 326 of the emission data portion 192 is gated into the gate 306, a fifth portion 328 of the emission data portion 192 is gated into the gate 308, and a sixth portion 330 of the emission data portion 192 is gated into the gate 310.

In the exemplary embodiment, the emission data acquired for the portion 192 is gated into a respective bin based on the motion state of the patient 16. Information to determine the motion state of the patient 16 may be acquired from, for example, the motion sensor 30. For example, the bin 300 may include emission data acquired at the beginning of the respiration phase (inspiration), and the bin 310 may include emission data acquired at the end of the respiration phase (expiration). Moreover, each intervening bin, e.g. bins 302, 304, 306, and 308 may include emission data that represents a motion state between inspiration and expiration. More specifically, each of the bins 300, 302, 304, 306, 308, and 310 are adapted to receive emission data that was acquired over a plurality of breathing cycles. Moreover, each of the bins 300, 302, 304, 306, 308, and 310 are adapted to receive emission data that represents approximately the same point in the patient's breathing cycle. Accordingly, each of the bins 300, 302, 304, 306, 308, and 310 include emission data representing a certain motion state of the patient 16. Thus, in the exemplary embodiment, the motion information acquired from the motion sensor 30 is utilized to divide the emission data 192 into six substantially equal portions and store the substantially equal portions in a respective bin 300, 302, 304, 306, 308, and 310.

In another exemplary embodiment, the information that represents emission data within the PET portion 192 may be binned or gated using a Quiescent Period Gating (QPG) algorithm or method. Quiescent as used herein refers to a respiratory state of relative inactivity, repose, and/or tranquility. The QPG algorithm may be implemented using, for example, the computer 44. The QPG algorithm performs quiescent period gating on the data subset 192 to account for the motion of a region of interest of the patient 16 based on a motion signal received from the motion sensor 30 shown in FIG. 1. More specifically, the QPG algorithm identifies the motion of the patient 16 and re-organizes the image data subset 192 to enable a motion-reduced image of the patient 16 to be reconstructed.

In operation, the QPG algorithm determines at least one quiescent period of at least a portion of the motion signal received from the motion sensor 30. The QPG algorithm utilizes the determined quiescent period to perform quiescent gating. For example, in one embodiment, the QPG algorithm utilizes the determined quiescent period to perform a displacement histogram-based gating of the image data subset 192. Specifically, the QPG algorithm divides the motion signal into intervals based on the displacement of the motion signal. The image data subset 192 is then gated into respective bins based on the displacement of the motion signal. Optionally, the QPG algorithm utilizes the determined quiescent period to perform a cycle-based gating of the image data subset 192. During operation, the QPG algorithm is configured to, extract image data from the image data subset 192 that corresponds to periods where, for each cycle, the motion signal is below or less than a predetermined threshold.

Referring again to FIG. 2, at 126 an auto-phase match procedure is implemented using the gated PET emission data formed at 124. In the exemplary embodiment, the auto-phase match procedure facilitates matching the CT image data with corresponding PET image data that has, been acquired during the same breathing phase based on the motion signal discussed above. In the exemplary embodiment, the PET data is a 4D gated data set. The gated PET images are then reconstructed in 126 using the phase-matched CT images as attenuation correction.

At 128, a reference gate is selected to further perform the motion correction 130 on the portion 192 to generate the hybrid PET portion 202. The reference gate may be selected manually by the operator. Optionally, the reference gate may be selected automatically by the computer 44. For example, the reference gate may be determined to be the bin 310 including information generated at the end of the respiration phase where the patient's diaphragm is at a highest point and the patient's lunge volume is a lowest point.

At 130, the gated PET images formed at 126 are corrected to substantially reduce or eliminate the effects of motion of the portion 192. In the exemplary embodiment, the motion correction is performed by registering the bins shown in FIG. 7 to the reference bin. More specifically, in the exemplary embodiment, the bins 302, 304, 306, 308 are registered to the bin 310 which was selected at 128. The gated bins 302, 304, 306, 308 may be registered to the reference bin 310, using either a rigid or non-rigid registration. The rigid and non-rigid registrations may be performed manually by the operator or automatically by the computer 44. In the exemplary embodiment, performing a non-rigid registration includes transforming the information within the bins 300, 302, 304, 306 and 308 in 3D space to align the information within the bins 300, 302, 304, 306 and 308 to the reference bin 310. For example, the images in the bin 300 may be slighted tilted with respect to the images in the reference bin 310. Accordingly, the images within the bin 300 are tilted to align the images with the images in the reference bin 310. The remaining bins 302, 304, 306 and 308 are also realigned to substantially match the images in the reference bin 310. In operation, the rigid registration process may be implemented by selecting anatomical or other features/points/landmarks and the images aligned using these feature or points along with detected edges or borders within the images. Alternatively, different markers may be used to identify known anatomical locations. The rigid registration also may be based on curved contours, for example, of bones within the image. The rigid registration may also be volume based or surface based. However, it should be appreciated that any rigid registration process may be performed that includes optimizing or calculating a certain comparable criteria or similarity measure.

In another embodiment, a non-rigid, or elastic, registration procedure may be utilized to perform the motion correction on the portion 192. In operation, the non-rigid registration includes non-rigid transformations. These non-rigid transformations allow local warping of image features and provide registrations that account for local deformations. Non-rigid transformation approaches include, for example, polynomial warping, interpolation of smooth basis functions (thin-plate splines and wavelets), and physical continuum models (viscous fluid models and large deformation diffeomorphisms), among others. The non-rigid registration is performed using the PET images forming the portion 192. The non-rigid registration may include, for example, warping of points or landmarks and providing a best fit along a contour with interpolation and correlation of the points or landmarks. Alternatively, a blending process may be performed that compares image voxels and blends corresponding regions. In general, the local non-rigid registration includes any type of elastic deformation model that allows for variations or movements in the different image sets. After the rigid or non-rigid registration process is completed, all of the bins 300 . . . 310 are averaged together. Specifically, the bins may be averaged together because each of the bins now represents the same spatial distribution of the counts. For example, a lesion that was in one location in a first gate and a second location in a second different gate, now appear to be in the same location in both gates.

As discussed above, the motion correction procedures are performed on the portion 192 to generate a hybrid portion 202 that is motion corrected. Thus, the hybrid portion represents motion-corrected or gated. PET images. As shown in FIG. 2, after the motion correction is completed and the hybrid portion 202 is generated, at 122, the hybrid portion 202 in combination with the information for portions 194 and 196 that were acquired from the initial un-gated PET imaging dataset 190, are re-inserted or combined with the non-motion corrected data to construct the hybrid image 200 shown in FIG. 2. Thus, the hybrid image 200 includes static (un-gated) portions (3D image volumes) that are not motion corrected, and at least one hybrid portion 202 that also represents at least one 3D image volume that has been motion corrected using 4D data.

Referring again to FIG. 2, as discussed above, at 112 at least one portion of the initial CT imaging dataset 160 acquired at 106 is utilized to, construct a hybrid image 170 (shown in FIG. 2) of the patient 16. In the exemplary embodiment, at 132 the CT images where several temporal images exist at the same axial location along the patient are sorted to form a gated CT image series. The number of gates is selected to match that performed for PET at 124. The reference gate 128 that is used for the PET motion correction is selected from the set of gated CT images 132 and used in 114 to form the image portion 172 of the hybrid volume 170.

In operation, the CT scan at 106 is performed by first identifying the location of potential motion in the patient 16. The area or areas having motion then may be scanned using a different protocol than areas not aftected by motion. In areas where motion is detected, the table 24 may be held in the same axial imaging position for a predetermined period of time. For example, on average it takes approximately 5 seconds per respiratory cycle. Accordingly, the table 24 may remain in the same position for 5-6 seconds to capture images at all parts of the cycle. This procedure may be accomplished for multiple table imaging positions. As a result of the CT imaging procedure, a relatively large quantity of CT images are generated. In the exemplary embodiment, at 132, the large quantity of CT images are sorted based on the breathing phase of the patient 16. More specifically, at least a portion of the CT images acquired at 106 are selected to be gated into bins based on the motion state of the patient 16. Information to determine the motion state of the patient 16 may be acquired from for example, the motion sensor 30.

In the exemplary embodiment, the reference gate selected at 128 is utilized to identify the gate of the CT images that are utilized to generate the hybrid CT portion 172. More specifically, the PET reference gate includes PET information acquired at a specific point or phase in the patient's breathing cycle. Moreover, the PET images at other gates are each motion corrected using the reference gate selected at 128. Therefore, the CT information acquired during the same respiration phase as information stored in the PET reference gate is utilized to form the CT hybrid portion 172. Thus, the reference gate 128 is utilized to select the portion of the CT imaging dataset that is affected by motion, e.g. portion 162. At 114, the motion selected CT information, e.g. the hybrid portion 172 formed at 132 is then reinserted or combined with the un-gated data to construct the hybrid CT image 170 that includes both 3D information for portions 164 and 166 and the hybrid portion 172 that represents the portion 162 after motion selection is performed.

A technical effect of the various embodiments described herein is to provide a fully or partially automatic steamlined 4D PET-CT workflow to generate a hybrid image volume or volumes. Various embodiments perform respiratory motion correction on PET-CT images utilizing gated 4D PET and gated 4D CT optionally with phase-match of the CT for PET attenuation correction. The 4D PET data is then input to a global non-rigid registration algorithm such that (N−1) gates are registered to the Nth gate, e.g. the reference gate. As a result, the quantity of respiratory motion induced blur in the PET images may be reduced and the quantification, as well as lesion detectability, is increased. Various embodiments are configured to generate the most clinically relevant information in a substantially automated manner, thus reducing the quantity of user interactions required and increasing the clinical efficiency.

Various embodiments described herein provide a tangible and non-transitory machine-readable medium or media having instructions recorded thereon for a processor or computer to operate an imaging apparatus to perform an embodiment of a method described herein. The medium or media may be any type of CD-ROM, DVD, floppy disk, hard disk, optical disk, flash RAM drive, or other type of computer-readable medium or a combination thereof.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do, not differ from the literal language of the claims, or the examples include equivalent structural elements with insubstantial differences from the literal language of the claims.

What is claimed is:

1. A method for generating a hybrid imaging volume, said method comprising:
acquiring a Positron Emission Tomography (PET) imaging dataset of an object using a PET imaging system, the PET imaging dataset including at least one motion affected portion and at least one non-motion affected portion;
identifying a motion affected portion of the PET imaging dataset;
motion correcting the identified portion of the PET imaging dataset to generate a hybrid portion; and
constructing a hybrid PET image volume using the hybrid portion and the at least one non-motion affected portion.

2. The method of claim 1 further comprising:
motion correcting at least one Computed Tomography (CT) imaging dataset portion using a PET reference bin to generate a hybrid CT portion; and
constructing a hybrid CT image using the hybrid CT portion and at least one non-motion affected CT portion.

3. The method of claim 1 further comprising:
gating the identified PET portion that is affected by motion dataset into a plurality of bins;
selecting at least one of the plurality of bins as a PET reference bin;
motion correcting a portion of the Computed Tomography (CT) imaging dataset based on the PET reference bin to generate a hybrid CT portion; and
constructing a hybrid CT image volume using the motion-corrected CT portion and at least one CT portion that is not affected by motion.

4. The method of claim 1 wherein motion correcting further comprises:
gating the identified PET portion that is affected by motion into a plurality of bins;
selecting a reference bin from the plurality of bins; and
registering the plurality of bins to the reference bin.

5. The method of claim 1 wherein motion correcting further comprises:
gating the identified PET portion that is affected by motion into a plurality of bins;
selecting a reference bin from the plurality of bins; and
performing at least one of a rigid registration and a non-rigid registration based on the selected reference bin.

6. The method of claim 1 wherein motion correcting further comprises:
gating the identified PET portion that is affected by motion into a plurality of bins using a Quiescent Period Gating (QPG) algorithm; and
using the QPG gated bin to generate the hybrid PET portion.

7. The method of claim 1 wherein motion correcting the PET portion further comprises:
utilizing a motion signal to identify the PET portion affected by motion;
determining a respiratory phase of at least a portion of the motion signal; and
mapping the identified PET portion into a plurality of bins based on the respirator phase of the motion signal.

8. The method of claim 1 wherein reconstructing further comprises reconstructing a two-dimensional (2D) hybrid PET image using a 3D hybrid portion and the at least one static image that is not affected by motion.

9. A method of improving the quality of a medical image, said method comprising:
generating a plurality of gated Positron Emission Tomography (PET) images:
motion correcting the gated PET images using a PET reference gate to generate a hybrid PET series of images;
selecting at least one Computed Tomography (CT) image having the same respiratory phase as the gated PET images stored in the PET reference bin; and
constructing at least one PET image volume using the hybrid PET series of images.

10. The method of claim 9 further comprising reconstructing at least one hybrid CT image using the selected CT image and at least one CT portion that is not affected by motion.

11. The method of claim 9 further comprising:
acquiring a Positron Emission Tomography (PET) imaging dataset of an object using a PET imaging system, the PET imaging dataset including at least one portion that is affected by motion and at least one portion that is not affected by motion;
identifying a portion of the PET images that are affected by motion to form a hybrid PET series of images; and
constructing a hybrid PET image using the hybrid portion and at least one PET image that is not affected by motion.

12. The method of claim 9 further comprising:
motion correcting a portion of the Computed Tomography (CT) imaging dataset based using a PET reference bin to generate a hybrid CT portion; and
constructing a hybrid CT image using the hybrid CT portion and at least one CT portion that is not affected by motion.

13. The method of claim 9 wherein generating the gated PET images further comprises:
identifying the PET images that are affected by motion dataset into a plurality of bins;
selecting at least one of the plurality of bins as the PET reference gate;
motion correcting a portion of the Computed Tomography (CT) images based on the PET reference gate to generate a hybrid CT image; and
inserting the hybrid CT image into a whole-body CT image that includes both the hybrid CT image and at least one image that is not affected by motion.

14. The method of claim 9 wherein generating a plurality of gated PET images further comprises:
identifying a portion of the PET images that are affected by motion;
gating the identified PET portion that is affected by motion into a plurality of bins to form the gated PET images;
selecting a reference bin from the plurality of bins; and
registering the plurality of bins to the reference bin.

15. The method of claim 9 wherein generating a plurality of gated PET images motion correcting further comprises identifying a portion of the PET images that are affected by motion; gating the identified PET portion into a plurality of bins; selecting a reference bin from the plurality of bins and motion correcting the gated PET images further comprises performing at least one of a rigid registration and a non-rigid registration based on the selected reference bin.

16. The method of claim 9 wherein generating a plurality of gated PET images motion correcting further comprises identifying a portion of the PET images that are affected by motion, and gating the identified PET portion into a single bin using a Quiescent Period Gating (QPG) algorithm.

17. The method of claim 9 wherein motion correcting the PET portion further comprises utilizing a motion signal to generate the gated PET images.

18. A multi-modality imaging system comprising a first modality unit, a second modality unit, and a computer operationally coupled to the first and second modality units, wherein the computer is programmed to:
acquire a Positron Emission Tomography (PET) imaging, dataset of an object using a PET imaging system, the PET imaging dataset including at least one motion affected portion and at least one non-motion affected portion;
identify the motion affected portion of the PET imaging dataset;
motion correct the identified portion of the PET imaging dataset to generate a hybrid portion; and
construct a hybrid PET image using the hybrid portion and the at least one non-motion affected portion.

19. A multi-modality imaging system in accordance with claim 18, wherein the computer is further programmed to:
motion correct a portion of the Computed Tomography (CT) imaging dataset based using a PET reference bin to generate a hybrid CT portion; and
construct a hybrid CT image using the hybrid CT portion and at least one non-motion affected CT portion.

20. A multi-modality imaging system in accordance with claim 18, wherein the computer is further programmed to:
gate the identified PET portion that is affected by motion dataset into a plurality of bins;
select at least one of the plurality of bins as a PET reference bin;
motion correct a portion of the Computed Tomography (CT) imaging dataset based on the PET reference bin to generate a hybrid CT portion; and
construct a hybrid CT image using the hybrid CT portion and at least one non-motion affected CT portion.
